(19) United States
(12) Patent Application Publication
(10) Pub. No.: US 2015/0355151 A1
(43) Pub. Date: Dec. 10, 2015

(54) SECURITY MARKER SYSTEMS AND METHODS WITH TRACKING SCENT

(71) Applicant: SmartWater Ltd, Liverpool (GB)

(72) Inventor: Michael Cleary, Liverpool (GB)

(73) Assignee: SMARTWATER LTD, Liverpool (GB)

(21) Appl. No.: 14/720,726

(22) Filed: May 22, 2015

Related U.S. Application Data
(60) Provisional application No. 62/001,860, filed on May 22, 2014.

(51) Int. Cl. G01N 33/00

(52) U.S. Cl. CPC G01N 33/0001

(57) ABSTRACT

A security marker for an item is disclosed which may include (i) an (optional) first component of the marker which is an indicator (e.g., identify the presence of the forensic component marker); (ii) a second component of the marker which is a forensic component defining a unique identification code or other information for the item, e.g., based on a combination of characteristics; and (iii) a chemical scent/trace, e.g., detectable only by a trained dog. Thus, the marker when deployed may advantageously be tracked/located, e.g., by a dog to aid in the tracing of items or personnel following an offence. Advantageously the chemical scent/trace may be detectible at a distance from the item.

### CROSS-REFERENCE TO RELATED APPLICATIONS

The subject application claims priority to U.S. Provisional Application No. 62/001,860, entitled “Improvements in the Tracking of Stolen Goods,” filed May 22, 2014, the contents of which are hereby incorporated herein. The present application also relates to U.S. application Ser. No. 14/714,168, entitled “Security Marker,” filed May 15, 2015, the contents of which are hereby incorporated herein.

### TECHNICAL FIELD

The present disclosure relates to security measures and in particular, security measures which may be used in the identification/authentication of goods so as to be able to, for example, distinguish original goods from counterfeit goods and/or to identify stolen goods or articles.

### BACKGROUND

There is an ever increasing level of crime in terms of the theft of valuable items. Goods not previously thought to be valuable are now also subjected to theft. In addition, profits from the production of counterfeit goods continues to rise. This is an international crime that has grown over recent years such that it is estimated to account for 6% of world trade and is increasing rapidly.

The use of a marker system in a court of law does require a guaranteed level of utility. Clearly, the reliability of any given marker system may be demonstrated through repeated successful outcomes within a criminal justice system. A system incorporating multiple independent markers, the analysis of which would provide increased verification of an authenticity of the marker.

Various measures have also been put into place to help distinguish between a genuine product and a counterfeit copy. Complex labeling is used in the form of branding and logo's as the first line of defense. However, the funds available to counterfeiters are such that they can use the highest quality printing methods to duplicate the branding themselves, so that the logo's and labels become counterfeit. In order to combat this, complex features that are difficult to copy have been added by manufacturers to make any copying more difficult. Such features include bar-coding, reflective and refractive inks and holograms.

However, these measures have also been copied and accordingly manufacturers have adopted the approach of adding markers to their products as a further way of deterring the production of counterfeits. Several different types exist that are both overt and covert. Overt markers are visible under normal lighting and can be designed in many different ways that are difficult to reproduce exactly. Others are covert and require some form of stimulation before they can be seen. These can also contain various features that are difficult to reproduce such as for example a random pattern.

A further type of marker system which may be used contains a covert fingerprint within the marker. One of the first such systems involved the use of DNA fingerprints containing artificially produced base sequences which provided a unique identification of the item to which it was applied. However, one weakness of DNA lies in its relative fragility and so this may be degraded through sunlight, heat or alkaline conditions.

The damage to a DNA molecule used as a security marker caused by exposure to aggressive radiation, heat, humidity, pH or chemicals will result in a failure to identify any fingerprint upon analysis. This could result in genuine items being classed as counterfeit and the legal ramifications and costs to both sides in the legal disputes that will follow will be enormous.

Moreover, existing security markers used in the identification of items are not particularly useful in assisting with the locating of the items (e.g., in the case of stolen goods). Rather, most products may employ separate measures for locating items, such as RFID technology, GPS enabled tracking, a trigerable audio alert, or the like. This, lack of comprehensive/combined identification and locating/tracking solutions inevitably contributes to added cost and unnecessary complexity.

Various systems are in place as security markers to deter theft. Some involve the use of a marker that is sprayed into protected areas that contain high value goods. In this type of system the marker is deployed automatically after detection of unauthorized access into specific areas within a building. These areas usually contain high value or easily re-sold goods such as cigarettes, perfumes or jewelry.

When such an automated marker system is deployed the goods being stolen and the personnel involved can be marked with a mixture that is unique to the premises from which the goods were stolen. Subsequent analysis of the marker from samples of clothing of those involved or from the goods themselves will allow both to be tracked back to the location from where they were stolen and the rightful owner can then be identified. Once the owner is identified then proceedings for theft can be started.

However, when the perpetrators leave the scene then tracking them can be difficult. Currently police rely on their knowledge of local problem areas in order to determine likely places to search for the stolen goods.

Thus, there exists a need for new and improved security marker systems and methods and, in particular, those that provide comprehensive/combined identification and locating/tracking solutions. These and other needs are met by the systems and methods disclosed herein.

### SUMMARY

The present disclosure is concerned with an improved security marker that may be used to recover stolen goods and in the authentication of goods so as to be able to, for example, distinguish original goods from counterfeit goods and to identify stolen goods or articles.

Therefore, according to a first aspect of the present disclosure there is provided a security marker for an item which may include (i) an (optional) first component of the marker which is an indicator (e.g., identify the presence of the forensic component marker); (ii) a second component of the marker which is a forensic component defining a unique identification code or other information for the item, e.g., based on a combination of characteristics; and (iii) a chemical scent/trace, e.g., detectable only by a trained dog. Thus, the marker when deployed may advantageously be tracked/located, e.g., by a dog to aid in the tracing of items or personnel following an offence. Advantageously the chemical scent/trace may be detectible at a distance from the item.

### BRIEF DESCRIPTION OF THE DRAWINGS

The above and other aspects, features and advantages of the disclosure will be apparent from the following more particular description of examples, as illustrated in the accompanying drawings, in which:

FIG. 1 depicts an exemplary security marker and related systems and methods, according to the present disclosure.

### DETAILED DESCRIPTION

A description of example embodiments of the present disclosure follows.

As described herein, a security marker including a forensic component may advantageously also include a volatile component such as a scent/tracer, e.g., that can be detected remotely such as by sniffer dogs. Preferably, the marker system may be substantially colourless under visible light and substantially odourless to human individuals.

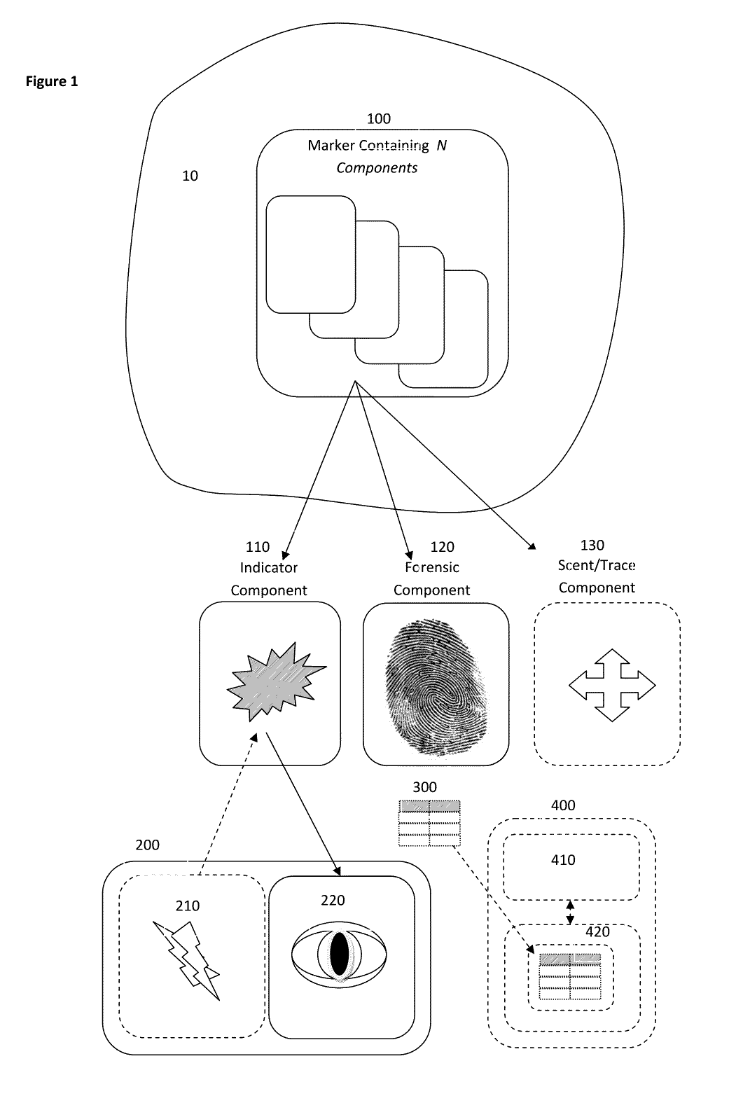

With reference to FIG. 1, an exemplary security marker 100 is depicted. Security marker 100 may advantageously be associated with an protected item 10, such as a consumer product requiring identification/authentication. In general, the security marker 100 may include a plurality of components. The N components of the marker 100 may represent the batch information for the marker and may be known/recorded at the time of creation of the marker 100.

In exemplary embodiments, the marker 100 may include an indicator component 110, a forensic component 120 and a volatile scent/tracer component 130. In some embodiments, an indicator component may not be employed. Advantageously the marker 100 may be configured such that an identification of the indicator component 110 may identify the presence or location of the forensic component 120. The forensic component 120 of the marker may typically define a unique identification code or other information for the item, e.g., based on a combination of characteristics. The volatile scent/tracer component 120 may be a chemical scent/trace, e.g., detectable only by a trained dog. Thus, the marker when deployed may advantageously be tracked/located, e.g., by a dog to aid in the tracing of items or personnel following an offence. In this way the marker 100 may advantageously provide both location/tracking information and identification information for the item 10.

In an example embodiments, a security system may be provided which includes, both a security marker 100, such as described herein, and an analysis tool 200 configured for enabling analysis of the indicator 110 and/or the forensic component 120 of the marker, e.g., in order to facilitate or enable identification of the signature. In further example embodiments, the security system may also include correlation information 300 adapted for facilitating or enabling identification of underlying data correlating to the signature, e.g., identification/owner information or the like.

In some embodiments, the analysis tool 200 may include a stimulation mechanism 210 for stimulating the marker such as to cause the cause the marker to exhibit an observable property/characteristic. In example embodiments, the observable property/characteristic may be hidden/less observable prior to the stimulation. Example properties/characteristics may include photo-responsive properties/characteristics (e.g., florescence, directionally specific reluctance patterns, etc.) thermo-response properties/characteristics (e.g., thermochromic), electro-responsive proprieties/characteristics, mechanically-responsive properties/characteristics or other types of properties/characteristics responsive to different types of stimuli. In further example embodiments, the analysis tool 200 may include an examination mechanism 220 for examining the marker, e.g., for qualifying or quantifying a signature of the forensic marker component 120, e.g., based on the stimulus. Thus, the examination mechanism may be configured for observing, qualifying and/or quantifying property/characteristics of the marker.

With reference still to FIG. 1, in some embodiments the correlation information 300 may operatively be associated with the analysis tool 200, e.g., so as to enable automatic correlation of a detected signature to the underlying data. Thus, in some embodiments, the correlation information 300 and analysis tool 200 may be operatively associated with processing system 400 including a processor 410 configured, e.g., via association with non-transient processor executable instructions, to enable the automatic identification of the underlying data correlating to the detected signature of the forensic marker component 120. In such embodiments the processor may further be associated with non-transient memory 420 adapted for storing (i) the processor executable instructions, (ii) the correlation information 300 and/or (iii) data received from the analysis tool 200, e.g., relating to, a signature of the forensic marker component. In other embodiments, the correlation information 300 may be provided as a reference to enable a user to correlate a detected signature to the underlying data.

The marker be deployed using any suitable deployment system which can deploy a liquid composition of the marker system. The marker system may be deployed as a liquid spray or as a fine mist depending on the circumstances of its use. For example, the marker may be housed in a pressurized container that is triggered either by virtue of a criminal perpetrator at a location for example by virtue of activating a triggering device such as a passive infrared detector. Alternatively, the device housing said marker system may be activated by an employee or such like at a store/shop or bank or the like during or after a criminal event has occurred. Once the perpetrator has been sprayed with the marker, they can then be tracked by trained sniffer dogs. Once the perpetrator is then identified and secured, an analysis of the marker system can take place. Since any given marker system will be unique for the location of the criminal event, this advantageously provides irrefutable proof in a court of law of the presence of the individual or item at the scene of the crime.

Although not evident to human senses the material can be detected by dogs trained to detect it. The material will be evident on the personnel involved and on the stolen goods. In the event that the perpetrators stay on foot then the scent will lead dogs directly to them and the stolen goods.

If the personnel involved use transport of some type then the scented material will be transferred to that. In most cases if a car is used then it has been stolen for the purposes. Again in most cases this is abandoned after the event. However it will be abandoned in an area close to the areas in which those involved reside. Alternatively the first car may be abandoned and the personnel and goods transfer to their own transport. The scented material would then be transferred to this vehicle.

The scented material is in addition to the existing indicator material that is present in the marker and this will also transfer from the clothing of the personnel and the stolen goods to the vehicles used.

The forensic marker preferably comprises an indicator material, which can quickly provide a preliminary, gross indication of the presence of a marker system according to the present disclosure. The indicator material can either be “overt” or “covert.” An overt material is typically one which can be seen unaided by technology, such as a dye or pigment. With an overt indicator, it is immediately evident from an observation of the article or person that a mark has been provide thereon which may act as a deterrent. In one embodiment both a covert and overt mark may be applied thus combining the deterrent effect of the overt mark with the covert properties of the covert mark. For example, if the overt mark failed to act as a deterrent and the perpetrator tried to remove the overt mark; even if they were successful the stolen item could nevertheless still be identified by virtue of the covert mark.

A covert indicator will remain hidden until some technical means or stimulus is used to make it obvious. Usually, a covert indicator will become visible upon application of a radiation source other than visible light, and of these, fluorescent indicators are most common. Thus, the covert indicator will often be at least one fluorescent material which is soluble in a solvent system, and which is easily detectable upon examination with ultraviolet light, for example. Alternatively or additionally, the indicator may comprise at least one phosphorescent material capable of phosphorescing when subjected to stimulus.

In terms of suitable indicators, both organic and inorganic materials were tested. Some organics, especially of the oxazinone functionality performed well, but did still degrade well below the required temperature and lost their fluorescence.

A compound, according to an example embodiment of the present disclosure, for use as an indicator is an inorganic emitter.

The product can be water or solvent based and can contain a dissolved or dispersed polymer solution or dispersion. Both are designed to allow transferability while the product is wet and then to form a clear, discrete surface coating when dry. The marker system preferably includes a matrix and an aqueous polymer emulsion to bind a marker to the surfaces of items, articles, goods, vehicles and/or premises. Advantageously, the polymer system, which may be water based to avoid the use of solvents, initially acts as an adhesive to secure the marker or surface coating to the goods being protected. As the goods may be subject to high temperatures, it may be desirable for the matrix to be able to withstand high temperatures; failing which, the matrix may lose its adhesion to the surface, by for example carbonising, and the marker system will simply fall off the surface, when the marker system is subjected, either directly or indirectly, to high temperatures. In order to ensure that a stolen item is identifiable even where it has been subjected to heat; it is desirable for the polymer emulsion and matrix combination to secure the marker system across a wide range of temperatures.

Various fingerprint technologies can be used singly or in combination with the preferred product containing at least two such technologies. The chemical and metallic fingerprints are based upon mixtures of components used only once and not repeated.

The fingerprint may comprise a solvent medium containing a volatile component, together with for example one or more trace materials which can be varied in such a manner as to produce unique formulations. The combinations of trace materials may advantageously be varied by modelling the compositions on, for example, binary strings to produce large numbers of unique products. However, other suitable coding methodologies may also be utilised as appropriate. The term “trace materials” applies herein to materials which would not normally be present in the environment of use. The most commonly used trace materials are metal compounds.

Trace materials can advantageously therefore be combined in a way which gives good evidential value to law enforcement agencies, as each unique formulation may be allocated to a particular premises, location or person, and this information is stored in a central database which can be accessed by a law enforcement agency receiving the report of a laboratory analyzing the mixtures which are to be discussed.

The trace materials may be assigned constant positions in a binary string with their presence being given by a “1”, and their absence by a “0”. If, for example, one were to set a limit of thirty digits for the string, one could begin with combinations of two trace materials, and generate all combinations containing any two trace materials. One could then go to groups of three trace materials, and generate all combinations of any three trace materials. This could continue until the number of trace materials is equal to the number of digits in the string.

With a thirty digit string, the total number of unique combinations of trace materials is approximately one billion. However, it is possible to prepare an infinite number of mixtures having compositions based upon unique binary sequences, the composition of each being unique.

Binary strings are provided as exemplary of the manufacturing procedures which can be used. Octal strings may also be used. Decimal numbers and random number generation can be used to generate potential codes, although these will need to be checked and converted to binary or octal sequences prior to use.

The unique nature of each composition can be checked during Quality Control following manufacture. The composition can then be stored in a database, allocated to a premises, location, or person, and the source of goods located at a later time can be traced to the premises, location or person via the composition.

Of course, the greater the number of trace materials used, the greater the certainty in identification later on, since the chance presence of trace materials can be ruled out.

In one embodiment of the present disclosure, inorganic materials may be used as the fingerprint. These materials have the best performance, of the materials tested.

A preferred fingerprint for use in the present disclosure is an organometallic material.

The present disclosure involves the use of a material in a marker system that can be detected by specially trained dogs, known a “sniffer” dogs. Marker systems are generally water based and so the materials to be detected by the dog's olfactory system will be water soluble.

Such materials are plentiful and several are already in use to assist in the training of the animals involved. Materials to be used are cheap and readily available, such things as sharks liver oil and vegetable oil are all examples. Some dogs have been estimated to have an olfactory sensitivity of one part per trillion or 1 in 10¹².

Suitable materials where tested that were both water soluble and were known to be easily detectable by suitably trained dogs. Specialist dog training companies exist that will train dogs to detect specific smells. Several of such were approached and undertook suitable training with a selection of dogs. As would be immediately evident to one of skill in the art, the present disclosure is not based upon detection of any specific material as dogs can be trained to detect anything that has a characteristic odour and accordingly any suitable chemical scent will suffice.

An additional material suitable for this role was added to the marker at a level of 1% and samples of the marker were supplied to the dog trainers. Samples of the concentrated material were also supplied to the trainers to be used in the training procedures.

Further to the training the results obtained showed a good level of detection with zero false positives. These were obtained from the stolen items themselves, from the individuals concerned and from their clothing. Results from secondary transfer were also good being higher than 90%. Secondary transfer involves the scent transferred from the surfaces onto which the spray fell originally to surfaces or regions of space to which they are moved. Such secondary surfaces/spaces include a car interior, car boot or room within a building.

Clearly in this type of situation the length of time between the application of the mark and the subsequent detection of the odour is an important variable as are the ambient temperature and air movement. The work undertaken indicated that given normal ambient conditions the scent remained detectable for a mean period of 1 month from being applied. This time is adequate in most circumstances and does provide the extra step that could assist in the tracking of stolen goods.

The teachings of all patents, published applications and references cited herein are incorporated by reference in their entirety.

While the present disclosure has been particularly shown and described with references to example embodiments thereof, it will be understood by those skilled in the art that various changes in form and details may be made therein without departing from the scope of the present disclosure encompassed by the appended claims.

1. A marker system for identifying/tracing items or personnel said marker comprising;
a forensic marker containing a unique code specific for the items/or their location
an indicator material to identify the presence of the forensic marker
a chemical scent detectable only by a trained dog
wherein the marker when deployed can be tracked by the dog to aid in the tracing of items or personnel following an offence.

2. The marker system of claim 1 wherein the chemical scent is substantially odourless to human individuals, non-toxic and substantially colourless under visible light.

3. A marker system of claim 1 wherein the indicator material comprises a fluorescent or phosphorescent material that is capable of fluorescing at particular wavelengths when subjected to a particular stimulus.

4. The marker as in claim 1 which is automatically deployed during the course of an offence.
